(19) United States
(12) Patent Application Publication
(10) Pub. No.: US 2008/0287971 A1
(43) Pub. Date: Nov. 20, 2008

(54) NEEDLE INSTRUMENTS AND IMPLANTABLE SLING ASSEMBLY; KITS COMPRISING THESE COMPONENTS; AND METHODS FOR USE

(75) Inventor: Richard P. Kuntz, Lakebay, WA (US)

Correspondence Address: SPECKMAN LAW GROUP PLLC, 1201 THIRD AVENUE, SUITE 330, SEATTLE, WA, 98101, US

(73) Assignee: GENERIC MEDICAL DEVICES, Seattle, WA (US)

(21) Appl. No.: 12/121,673

(22) Filed: May 15, 2008

Related U.S. Application Data
(60) Provisional application No. 60/938,174, filed on May 15, 2007.

(51) Int. Cl. A61B 17/00

(52) U.S. Cl. 606/151

(57) ABSTRACT

Insertion instruments for implanting various types of biocompatible slings and sling assemblies for repair, reconstruction and repositioning of internal anatomical structures are provided. In general, the insertion instruments have an a curved needle portion mounted at a distal end of a handle, wherein the handle has opposing side surfaces that are convexly raised with respect to adjacent grip surfaces, and each of the opposing side surfaces is provided with a recess having a generally thumb-like configuration positioned toward the center of the handle from the distal end of the handle.

### CROSS REFERENCE TO RELATED APPLICATION

This application claims priority under 35 U.S.C. §119(e) to U.S. Provisional Patent Application No. 60/938,174, filed May 15, 2007.

### FIELD OF THE INVENTION

The disclosed invention relates to instruments, components, assemblies, and kits for implanting slings, supports, and other implantable devices for treating conditions such as urinary incontinence, vaginal prolapse, hernias, and the like.

### BACKGROUND OF THE INVENTION

The placement of urinary slings and similar types of devices using surgical and minimally invasive surgical techniques is well known. Many different procedures are used and have been described in the prior art literature. The following patents and patent publications describe various techniques and procedures for treating urinary incontinence, vaginal prolapse, and related conditions: U.S. Pat. Nos. 5,899,909, 6,273,852, 6,638,211, 6,612,977, 6,911,003, and 7,070,556; U.S. Patent Publication Nos. 2002/0165556A1 and 2005/0148813A1. The following literature references describe various techniques and procedures for treating urinary incontinence, vaginal prolapse, and related conditions: Rackley, et al., “Tension-free Vaginal Tape and Percutaneous Vaginal Tape Sling Procedures” in Techniques in Urology, Vol. 7, No. 2, pp. 90-100 2001; “TVT-Tension free Transvaginal Tape” http://www.urogynecologychannel.net/tvt3.php 2003; Kohli, et al. “Tension-free Vaginal Tape: A Minimally Invasive Technique for Treating Female SUI” in Contemporary OB/GYN pp. 141-164 May 1999; Rutman, et al. “Long-Term Durability of the Distal Urethral Polypropylene Sling Procedure for Stress Urinary Incontinence: Minimum 5-Year Followup of Surgical Outcome and Satisfaction Determined by Patent Reported Questionnaires” The Journal of Urology Vol. 175, pp. 610-613 February 2006; Tash, et al. “Artificial Graft Slings at the Midurethra: Physiology of Continence” Current Urology Reports 2003; Rodriguez, et al. “Prospective Analysis of Patients Treated with a Distal Urethral Polypropylene Sling for Symptoms of Stress Urinary Incontinence: Surgical Outcome and Satisfaction Determined by Patient Driven Questionnaires”, The Journal of Urology Vol. 170, pp. 857-863 September 2003; Mourtzinos, et al. “Transobturator Versus Retropubic Suburethral Tapes for Stress Urinary Incontinence” Nature Vol. 3, No. 2 pp. 62-63 Feb. 2006; Madjar, et al. “Urethral Erosion of Tension-Free Vaginal Tape”, Urology Vol. 59, No. 4 2002. These patent and literature references are incorporated herein by reference in their entireties.

Various types of instruments, sling devices, and the like have also been developed for use in the techniques and procedures described above. The following patents and publications describe various surgical instruments and assemblies for use in surgical and minimally invasive surgical methods for treating urinary incontinence, vaginal prolapse, and related conditions: U.S. Pat. Nos. 6,491,703, 6,612,977, 7,070,556; U.S. Patent Publications 2006/199994A1, 2005/0277807A1, 2006/063968A1, 2003/176875A1, 2005/148813A1, 2006/015001A1, 2005/075660A1, 2005/131392A1, 2004/097974A1 and 2004/225181A1, for example. These references are incorporated herein by reference in their entireties.

### SUMMARY OF THE INVENTION

Instruments, components, assemblies, and kits for implanting slings and other implantable materials and devices are disclosed. Several surgical instruments and assemblies are described, including: a gently curved needle instrument; a generally uniformly curved, bidirectional needle instrument; a pair of complementary, directional, curved needle instruments; a sling and sleeve assembly; a sling/sheath and sleeve assembly; and kits comprising two or more of the above needle instruments in combination with a sling and sleeve assembly. In one embodiment, kits of the present invention provide an assembly of sterile, single use components for use as a suburethral and/or bladder neck sling indicated for treatment of female stress urinary incontinence (SUI) resulting from either hypermobility, intrinsic sphincter deficiency, or both. One of the advantages of kits of the present invention is that multiple insertion instruments are provided in combination with a sling assembly that is mountable on multiple configurations of insertion instruments, permitting the medical professional to choose appropriate instruments and combinations for carrying out different interventional procedures for placement of the sling assembly, depending on the patient's anatomy, condition, and the like.

The sling assembly comprises a length of porous material, such as a length of a synthetic mesh piece, and an optional sheath covering the mesh. The sling assembly is mounted on or mountable on or associated, at each end, with a sleeve designed to be mounted on a needle instrument(s) prior to or during an interventional procedure. The sleeves may have strategically placed slots that permit mounting on needle instruments having different configurations, thereby providing a common sling assembly mountable on different needle instruments for carrying out different procedures. The needle instruments have a handle configuration designed for convenient and ergonomic use in placement of the sling. Detailed descriptions of the instruments, components, assemblies and kits are provided below with reference to the drawings.

### BRIEF DESCRIPTION OF THE DRAWINGS

The disclosed invention will be described in greater detail in the following detailed description, with reference to the accompanying drawings, wherein:

FIGS. 1A-1D show an embodiment of a gently curved needle instrument of the disclosed invention, with FIG. 1A showing a perspective view, FIG. 1B showing a side view, FIG. 1C showing a view rotated 90° from the view of FIG. 1B, and FIG. 1D showing a top view;

FIGS. 2A-2D show a generally uniformly curved, bidirectional needle instrument of the disclosed invention, with FIG. 2A showing a perspective view, FIG. 2B showing a side view, FIG. 2C showing a view rotated 90° from the view of FIG. 2B, and FIG. 2D showing a top view;

FIGS. 3A-3D show a first (right hand) directional, curved needle instrument of the disclosed invention, with FIG. 3A showing a perspective view, FIG. 3B showing a side view, FIG. 3C showing a view rotated 90° from the view of FIG. 3B, and FIG. 3D showing a top view;

FIGS. 4A-4D show a second (left hand) directional, curved needle instrument of the disclosed invention, with FIG. 4A showing a perspective view, FIG. 4B showing a side view, FIG. 4C showing a view rotated 90° from the view of FIG. 4B, and FIG. 4D showing a top view;

FIGS. 5A-5D show another embodiment of a gently curved needle instrument of the disclosed invention, with FIG. 5A showing a perspective view, FIG. 5B showing a side view, FIG. 5C showing a view rotated 90° from the view of FIG. 5B, and FIG. 5D showing a top view;

FIG. 6 shows a perspective schematic view of an embodiment of a sling assembly of the disclosed invention;

FIG. 7A shows an embodiment of a sleeve component of the present invention, and FIG. 7B shows an enlarged view of the interface region of the sleeve component of FIG. 7A;

FIG. 8 illustrates a schematic view of the sleeve component associated with the sling assembly and mounted on a gently curved needle instrument of FIGS. 1A-1D;

FIG. 9A illustrates a schematic view of the sleeve component associated with a sling assembly in position for mounting in a first orientation on the directional, curved needle instrument of FIGS. 3A-3D, FIG. 9B shows the sleeve component associated with the sling assembly in position for mounting in a first orientation on the directional, curved needle instrument of FIGS. 4A-4D, FIG. 9C shows the sleeve and sling assembly mounted in the first orientation on the directional curved needle instrument illustrated in FIG. 9A, and FIG. 9D shows the sleeve and sling assembly mounted in the first orientation on the directional curved needle instrument illustrated in FIG. 9B, with portions of the sling assembly omitted for purposes of clarity.

FIG. 10A illustrates a schematic view of the sleeve and the sling assembly mounted in a second orientation on a directional, curved needle instrument, and FIG. 10B shows the sleeve and the sling assembly mounted in the second orientation on another directional, curved needle instrument, with portions of the sling assembly omitted for purposes of clarity; and

FIG. 11 illustrates a schematic view of a packaged kit comprising a plurality of needle instruments and the sling assembly of the disclosed invention.

### DETAILED DESCRIPTION OF THE INVENTION

Needle instruments, components, and assemblies of the disclosed invention may be used separately or in combination for implanting various types of implantable devices, slings, meshes, tapes, tissues, fibers, or the like for repair, reconstruction, and repositioning of various anatomical structures and placement of various instruments and devices. One advantage of the needle instruments, components, and assemblies of the disclosed invention is their versatility and their usefulness in a variety of surgical and minimally invasive procedures. In particular, sling assemblies of the present invention may be mounted on or used in combination with a variety of insertion instruments suitable for carrying out many types of interventional procedures.

The term “distal” is used herein to refer to the direction toward the free end of the needle or insertion instrument and away from the free end of the handle; the term “proximal” is used herein to refer to the direction toward the free end of the handle and away from the free end of the needle or insertion instrument. Similarly, when used with reference to a sleeve or sling assembly including a sleeve, the term “distal” refers to the direction toward a free end of the sleeve and away from a central area of the sling; and the term “proximal” refers to the direction toward the central area of the sling and away from the free end of the sleeve.

In general, the insertion instruments described herein have a handle and a needle component and may be designed for single use and disposal following use, or they may be constructed from a material that can be autoclaved or otherwise re-sterilized, allowing multiple uses of the instruments. Suitable autoclavable or re-sterilizable materials are well known in the art. The term “needle” is used herein to refer to a substantially rigid, rod-like structure having a curved configuration along at least a part of its length. Needle structures of the disclosed invention may have a substantially constant diameter along their lengths, or a variable diameter along all or a portion of their lengths. The curved section may be uniformly curved or irregularly curved. Needles of the disclosed invention may have a “pointed” distal end or a blunt or chamfered or rounded distal end. The needle structures of the disclosed invention are used as instruments for guiding devices or materials to anatomical locations but are not necessarily sharp and are not necessarily capable of penetrating intact tissue on their own.

Handles of the disclosed invention may have a regular configuration, an irregular configuration, or a complex, smooth, curved configuration that is ergonomic and facilitates handling and guidance of the needles. The handles are preferably substantially rigid, although softer or resilient materials may be incorporated in a rigid framework or supporting structure to provide comfortable and positive handling. Materials such as metals (stainless steel and the like), metallic alloys, ceramics, cer-met materials, plastics, thermoplastic polymers, substantially rigid rubber and rubber-like materials and the like may be used for the construction of handle and needle components. The materials are preferably biocompatible, substantially non-corrosive, and can be sterilized using autoclaving, radiation, or other techniques.

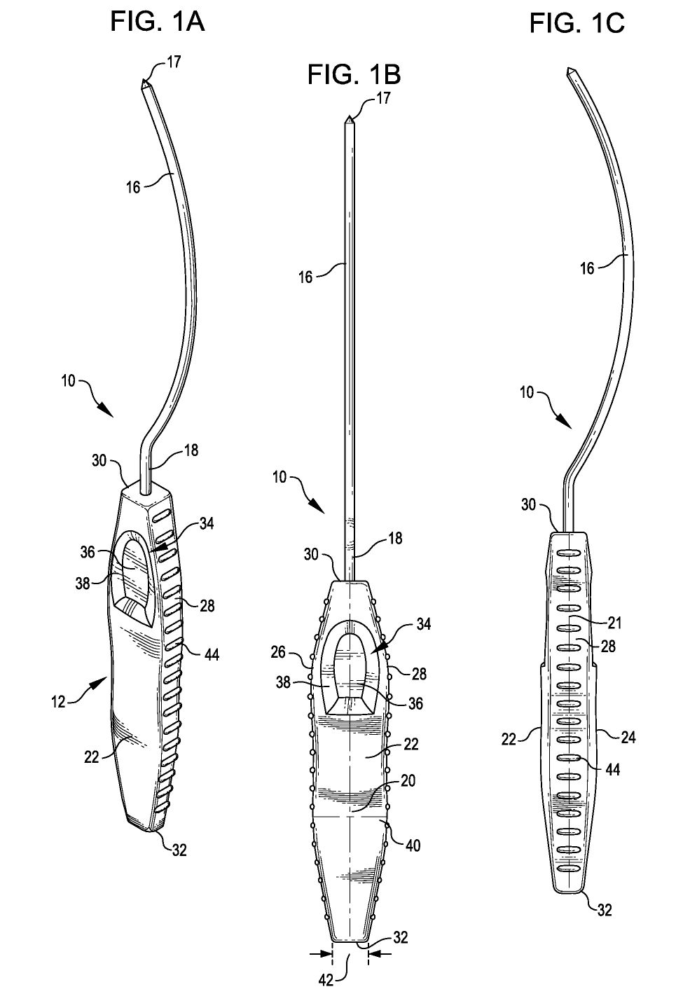

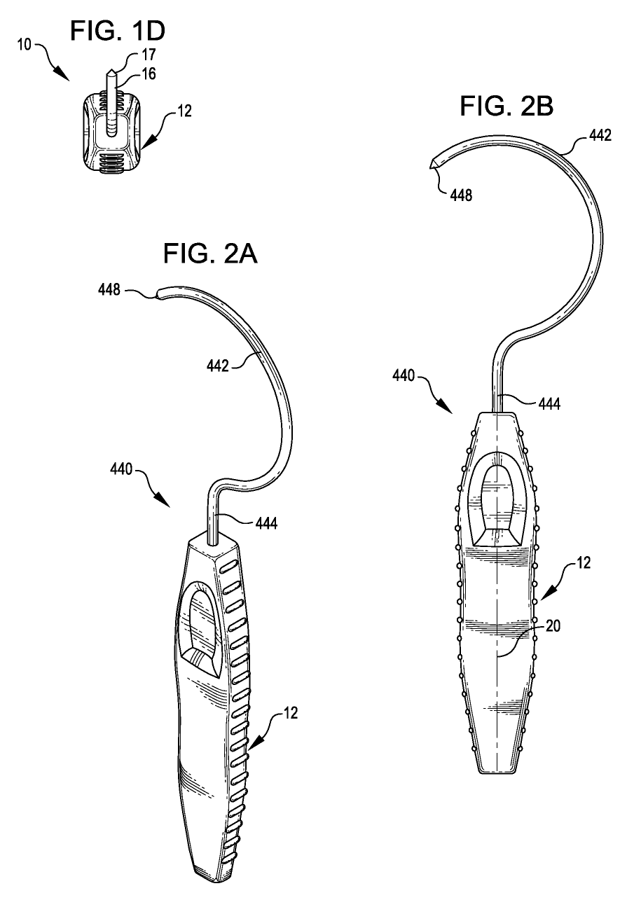

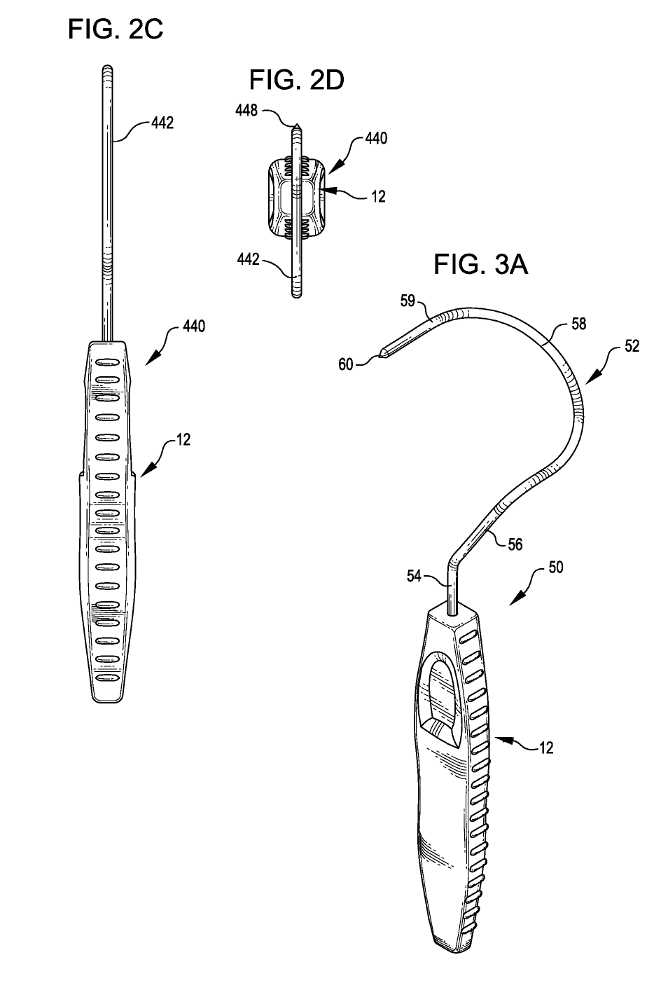

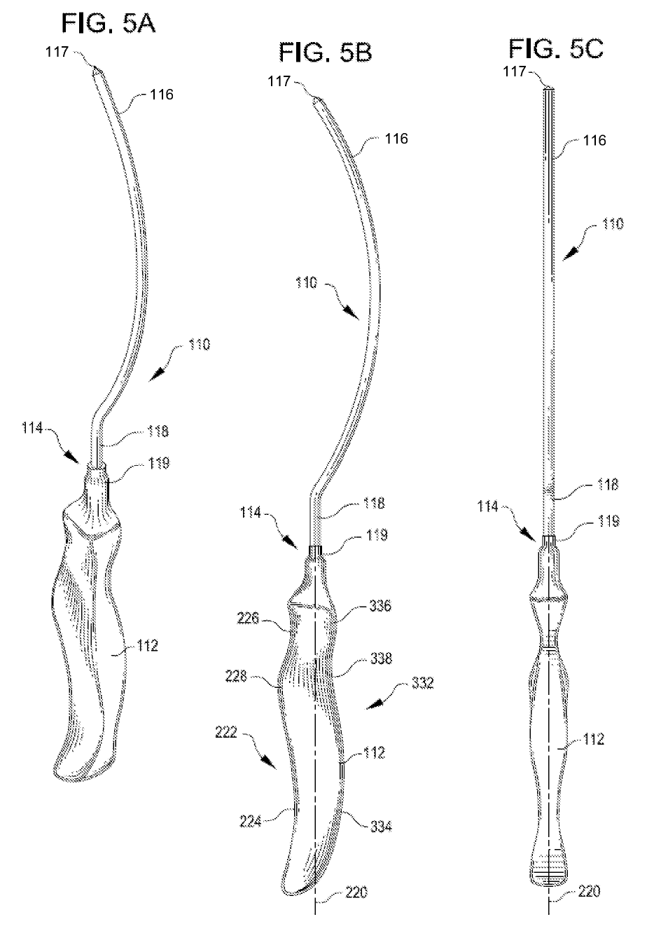

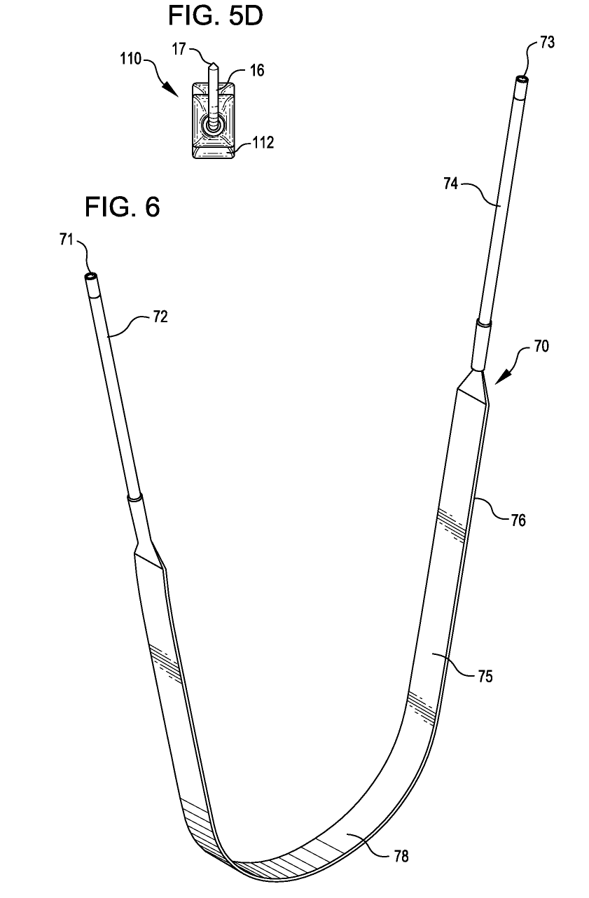

One handle configuration is described in detail with reference to FIGS. 1A-D, which illustrate a gently curved needle insertion instrument 10 of the disclosed invention. The insertion instruments illustrated in FIGS. 2A-2D, 3A-3D, and 4A-4D may comprise similar handles, as illustrated, and may be constructed in generally similar fashions. An alternative handle configuration is shown in FIGS. 5A-5D. It will be apparent to one of ordinary skill in the art that needle components of the disclosed invention may be used with different handle structures and, similarly, that handle structures of the disclosed invention may be adapted for use with different types of insertion instruments and needle configurations. The scope of applicant's inventions is limited only by the appended claims.

In the embodiment illustrated in FIGS. 1A-1D, insertion instrument 10 comprises a handle 12 and a curved needle 16. An aperture is provided at a distal end 30 of handle 12 to receive a proximal needle section 18 of curved needle 16 for rigidly mounting the needle to the handle 12. Proximal needle section 18 may be oriented in an axially vertical orientation and substantially aligned on a central axis 20 of handle 12, as illustrated, or it may be aligned at an angle with respect to central axis 20. The needle may be mounted to handle 12 in a fixed, non-adjustable manner. Alternatively, the needle may be mounted in handle 12 detachably and/or adjustably using a screw mount, bayonet mount, or a similar detachable mounting system.

Handle 12 comprises a first side surface 22, a second side surface 24, a first grip surface 26, a second grip surface 28, a distal end 30, and a proximal end 32. In the embodiment illustrated in FIGS. 1A-1D, first and second side surfaces 22 and 24, respectively, first and second grip surfaces 26 and 28, respectively, and distal and proximal ends 30 and 32, respectively, are generally substantially similar to one another and aligned in substantially the same orientation with respect to the central axis 20. Handles 12 may be provided as a unitary component or, in some embodiments, handles 12 may be assembled by joining two substantially identical handle pieces along a joint 21 substantially bisecting the first and second grip surfaces 26 and 28. In alternative embodiments, first and second side surfaces 22 and 24, first and second grip surfaces 26 and 28, and distal and proximal ends 30 and 32, may be asymmetrical with respect to one another and may be aligned asymmetrically with respect to central axis 20.

As illustrated in FIG. 1C, side surfaces 22 and 24 are generally convexly curved, or raised, with respect to the edges of first and second grip surfaces 26 and 28. Side surfaces 22 and 24 have generally narrower, tapered portions in proximity to distal and proximal ends 30, 32 and generally wider portions at distal and proximal end regions located toward the center of side surfaces 22 and 24 with respect to distal and proximal ends 30, 32. The dimensions of distal end 30 and proximal end 32 may be substantially the same, as illustrated in FIG. 1B. In alternative embodiments, the dimensions of distal end 30 and proximal end 32 are different from one another.

Handle 12 is designed to facilitate gripping and to provide a convenient and ergonomic configuration for holding and manipulating the insertion instrument during an interventional procedure. In the handle embodiment illustrated in FIG. 1B, for example, the widest portions of side surfaces 22 and 24 are generally in the area of recess 34 in the distal end region of the handle 12 and in the area of proximal end region 40. In one embodiment, the widths of side surfaces 22 and 24 in the area of recess 34 and proximal end region 40 are substantially equal. In another embodiment, side surfaces 22 and 24 of the handle narrow between the widest portions in the distal and proximal end regions to provide a slightly narrower, or “waisted” central handle portion; in alternative embodiments, the widths of side surfaces 22 and 24 are substantially constant between the distal and proximal end regions.

In the embodiment of handle 12 illustrated in FIGS. 1A-1D, beginning at the distal end 30, the edges of side surfaces 22 and 24 curve away from central axis 20 for about ¼ of the total length until they reach a widest point at the distal handle region, and then curve slightly toward central axis 20 at a central region of handle 12. The side surfaces then curve away from central axis 20 until they reach a widest point at the proximal handle region 40, and then curve toward central axis 20 and proximal end 32. In this embodiment, the widest portion of the handle 12 at the distal and proximal end regions of side surfaces 22 and 24 measure about twice the width (42) of distal and proximal ends 30 and 32.

One or both of the handle side surfaces 22, 24, is optionally provided with a recess 34 having a generally thumb-like configuration located at a distal region and positioned toward the center of the handle from distal end 30. Recess 34, in the embodiment shown, has a central depression 36 surrounded by a tapered perimeter wall 38. In alternative embodiments, the perimeter wall may be curved. The surface of depression 36 may be generally planar and aligned on or at an angle to central axis 20, or it may be curved in a regular or irregular pattern. In some embodiments, the surface of depression 36 may be generally smooth, while in other embodiments, the surface of depression 36 may be provided with discontinuities or raised portions that provide frictional surfaces during holding and manipulation of the handle. The overall length of recess 34 is generally at least about 10% of the total length of each side surface and, in some embodiments, may be from about 15% to about 25% of the total length of each side surface. The maximum depth of recess 34 is preferably from about 1 mm to about 1 cm. In a preferred embodiment, recesses may be provided on both handle side surfaces 22, 24, in substantially the same locations.

In the embodiment illustrated in FIGS. 1A and 1B, depression 36 and perimeter wall 38 have a curved portion extending toward handle distal end 30 and a generally linear base portion oriented closest to the center of handle 12. In alternative embodiments, the recess may have generally linear proximal and distal base portions, joined by generally linear or curved side portions, presenting a more square or rectangular overall configuration that provides a comfortable depression for placement of the user's thumb.

Grip surfaces 26, 28 are continuous with or generally adjacent to the edges of side surfaces 22, 24 and the three dimensional profile of grip surfaces 26, 28 consequently matches the edges of side surfaces 22, 24. Grip surfaces 26, 28 have a generally smaller width than the width of side surfaces 22, 24 in regions located centrally of distal and proximal ends 30 and 32 and, in the embodiments illustrated, the width of grip surfaces 26, 28 is substantially constant along their length. The width of grip surfaces 26, 28 may be generally similar to the width 42 of proximal and distal ends 30, 32 of side surfaces 22, 24.

Each grip surface, one of which is illustrated as grip surface 28 in FIG. 1C, is optionally provided with a plurality of raised grip elements 44. Grip elements 44 are generally oriented substantially transverse to the central axis 20 of handle 12, but they may be oriented at various angles to central axis 20 as well. Grip elements 44 may have a generally longitudinal, elongated oval configuration, as shown or, in alternative embodiments, grip elements may be generally rounded, curved, polygonal, or the like. Grip elements 44 may be provided extending substantially along the length of grip surface 28, as shown in FIG. 1C, or grip elements may be provided intermittently along grip surface 28, or in only one or more isolated regions of the grip surface 28. In one embodiment, the grip elements 44 are uniformly distributed along grip surface 28, having substantially the same distance between each grip element 44.

Various configurations of insertion instruments, needles, and other types of instruments may be used in combination with handles of the present invention. As shown in FIGS. 1A-1D, needle 16 is gently curved and forms an arc of from about 40° to about 90°. In one embodiment, needle 16 forms an arc of from about 60° to about 70°. Needle 16 is “bi-directional” in the sense that it may be used in a right-handed or left-handed orientation. In some embodiments, as illustrated, needle 16 has a length that is substantially similar to, or greater than, the length of handle 12 and any transition or mounting portion forming part of handle 12. In one embodiment, the overall length of needle 16 is at least 110% greater than the overall length of handle 12. Needle 16 is curved convexly with respect to central axis 20 and the distal end 17 of needle 16 points toward first side surface 22. The curve is gentle and substantially constant. In some embodiments, such as the embodiment illustrated in FIGS. 1A-1D, both the proximal end and distal end 17 of the curved portion of needle 16 terminate substantially on central axis 20. Curved needle 16 is preferably substantially planar, as illustrated in FIG. 1D.

FIGS. 2A-2D illustrate another bidirectional, curved needle instrument 440 of the disclosed invention having a handle 12 similar to the handle described above. Curved needle section 442 extends from a proximal end where it is integral with or joins a straight transition section 444 that, in the embodiment illustrated, is substantially aligned on central axis 20. Curved section 442 curves along a generally constant radius around a center point on or proximate central axis 20. In one embodiment, as shown, the distal terminal end 448 or curved needle section 442 extends past a centerline formed by central axis 20 and forms an arc of from about 5° to about 85° beyond the centerline and, in some embodiments, forms an arc of from about 20° to about 70° beyond the centerline. In one embodiment, curved needle section 442 forms an arc of about 45° extending beyond the centerline. Curved needle section 442 is preferably substantially planar and may be aligned on a plane that substantially bisects the first and second handle surfaces. As illustrated in FIG. 2D, in some embodiments, curved needle 442, or a portion of curved needle 442, may be aligned on a plane that is angled with respect to central axis 20 of handle 12.

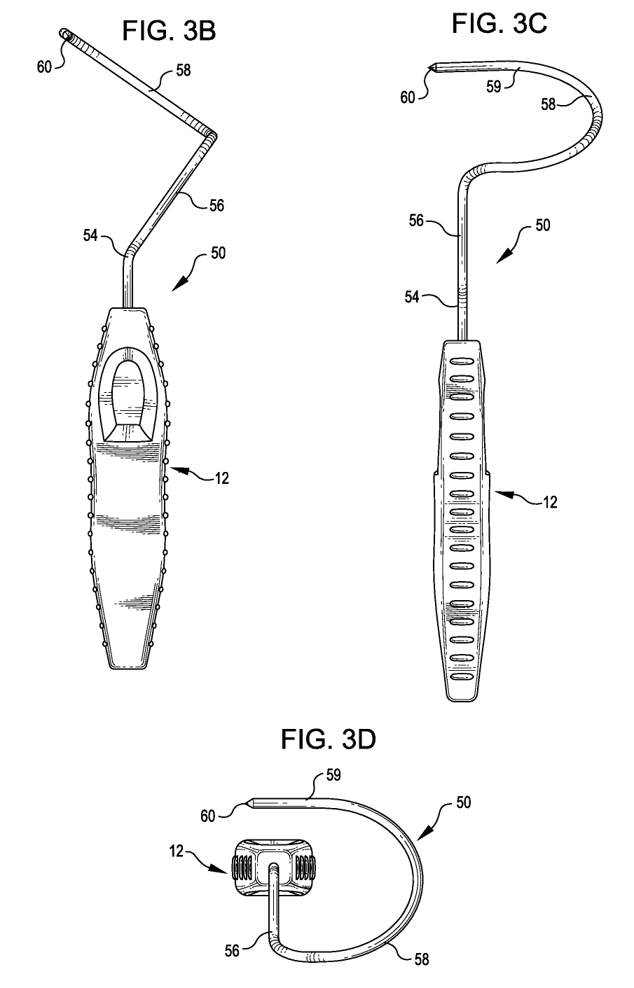

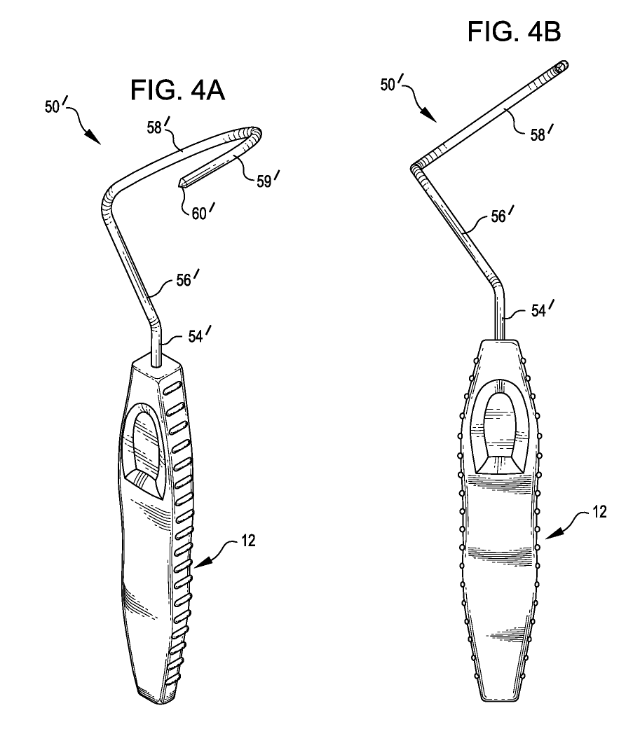

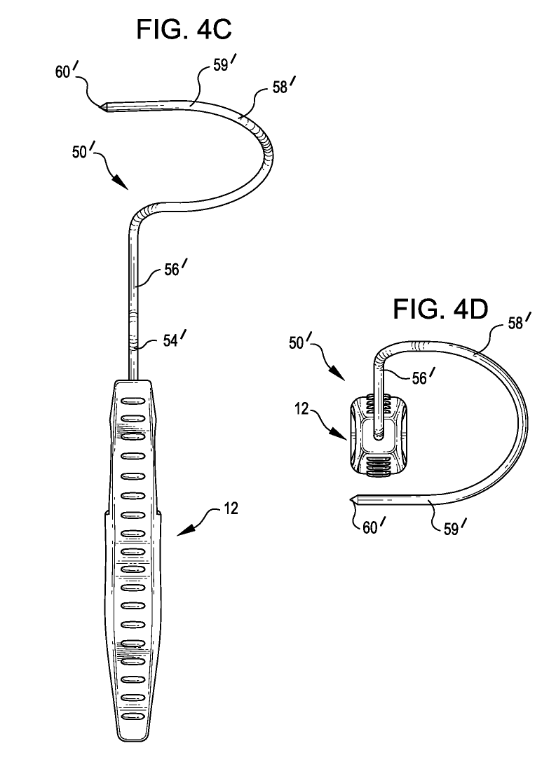

FIGS. 3A-3D illustrate a directional, curved needle instrument 50 of the disclosed invention having a handle similar to the handles described above. FIGS. 4A-4D illustrate another directional, curved needle instrument 50′ of the disclosed invention having a similar configuration to that of the directional, curved needle 50 illustrated in FIGS. 3A-3D and having corresponding elements numbered correspondingly. Directional needles 50 and 50′ may be used as right-hand and left-hand curved needles and are oriented and geometrically arranged for complementary, right-handed and left-handed use. Directional needles 50 and 50′ are generally provided in a substantially mirror image arrangement with respect to one another.

Curved needle section 52 extends from a proximal end where it is integral with or joins a transition section 54 that, in the embodiment illustrated, is substantially aligned on central axis 20. Extending distally from transition section 54, curved needle section 52 has an angled portion 56 that transitions into a curved portion 58 and terminates at distal terminal end 60. In some embodiments, there may be a transition section in proximity to the junction of angled portion 56 and curved portion 58 having a substantially straight alignment. The axis of angled portion 56 extends at an angle of from about 10° to about 80° from the central axis of transition section 54 and, in some embodiments, extends at an angle of from about 15° to about 55° from a central axis of transition section 54. In one embodiment, the axis of angled portion 56 extends at an angle of about 35° from the central axis of transition section 54. Angled portion 56 may form a straight line, as illustrated, or may be very gently curved.

Angled portion 56 then transitions into curved section 58 which, in the embodiment illustrated, curves through an arc of about 180° and has a distal extension 59 that terminates at distal terminal end 60. Distal extension 59 is, in one embodiment, a straight extension of the distal curved section 58 that extends beyond the axis of angled portion 56. In alternative embodiments, distal terminal end 60 may be positioned substantially at the 180° arc, or at a point at which curved section 58 forms less than an arc of 180°. Curved section 58 is preferably aligned on a plane and does not form a spiral or helical structure. The plane of curved section 58 is preferably arranged at an angle of from about 40° to about 130° from the plane of angled portion 56 and, in another embodiment, is arranged at an angle of from about 70° to about 110° from the plane of angled portion 56. In one embodiment, the plane of angled portion 56 extends at an angle of about 90° from the plane of transition section 54.

Various mounting orientations may be used for instruments of the disclosed invention. In general, the orientations illustrated are preferred for many types of procedures. The needle instruments 10 and 440 are bi-directional, in the sense that when the needle instrument 10 is rotated 180° about its central axis, the needle orientations are mirror images of one another. The directional needle instruments 50 and 50′, illustrated in FIGS. 3A-3D and FIGS. 4A-4D, respectively, may be provided as a complementary pair of directional needle instruments.

FIGS. 5A-5D show another embodiment of an insertion instrument 110 of the disclosed invention, provided with handle 112. Instrument 110 comprises handle 112, an intermediate transition section 114, and a curved needle 116 as illustrated in FIGS. 5A-5D. In general, all or part of transition section 114 is formed integrally with or is mounted or attached rigidly to curved needle 116 and handle 112. In the embodiment illustrated in FIGS. 5A-D, curved needle 116 is integrally formed with or mounted on a proximal straight needle section 118 forming part of transition section 114. Proximal needle section 118 may be oriented in an axially vertical orientation and substantially aligned on central axis 220, as illustrated, or it may be aligned at an angle with respect to central axis 220. Transition section 114 may also incorporate a mounting stem 119 formed integrally with or mounted on a distal end of handle 112. Mounting stem 119 may, similarly to proximal needle section 118, be oriented in an axially vertical orientation and aligned on central axis 220, or it may be aligned at an angle with respect to central axis 220. Mounting stem 119 may be sized to receive proximal needle section 118 for rigidly mounting the needle to the handle. The mounting stem may be tapered, or chamfered, having a smaller diameter closer to its terminal end where it joins proximal needle section 118. The needle may be mounted to the handle in a fixed, non-adjustable manner. Alternatively, the needle may be mounted in the handle detachably and/or adjustably using a screw mount, bayonet mount, or a similar detachable mounting system.

Handle 112 has a central axis 220, substantially aligned with a central axis of transition section 114, and a complex curved configuration. As illustrated in FIG. 5B, first and second surfaces 222, 332, respectively, of handle 112, are substantially opposite one another with respect to central axis 220, and the curved configurations of the first and second surfaces of handle 112 are different from one another. The handle surface configuration is thus not symmetrical with respect to central axis 220. The proximal end of handle 112 is “off-axis” and terminates toward the first surface of the handle, as illustrated.

One curved surface, illustrated as a first surface 222 in FIGS. 5A-B, has a proximal, generally concave curved surface 224 separated from a distal, generally concave curved surface 226 by an intermediate protrusion 228. The proximal curved surface 224 generally has a longer length than the distal curved surface 226, and generally incorporates more than 50% and, in some embodiments, more than 60% of the total length of handle 112. The distal curved surface 226 has a generally shorter length than the proximal curved surface 224, and generally incorporates less than 40% and, in some embodiments, less than 30% of the total length of handle 112. The apex of intermediate protrusion 238 is located toward the distal end of handle 112 and is preferably distal to a midline of the length of handle 112.

Another curved surface, illustrated as a “second” surface 332, is generally opposite first surface 222. Second surface 332 has a proximal, generally convex curved surface 334 separated from a distal, generally convex curved surface 336 by an intermediate depression 338. The proximal curved surface 334 generally has a longer length than the distal curved surface 336, and generally incorporates more than 50% and, in some embodiments, more than 60% of the total length of handle 112. The distal curved surface 336 has a generally shorter length than the proximal curved surface 334, and generally incorporates less than 40% and, in some embodiments, less than 30% of the total length of handle 112. Intermediate depression 338 is located toward the distal end of handle 112 and is preferably distal to a midline of the length of handle 112. The nadir of intermediate depression 338 is also, in some embodiments, distal to a midline of the length of handle 112 and may additionally be distal to the apex of intermediate protrusion 228. In some embodiments, the apex of intermediate protrusion 228 and the nadir of intermediate depression 338 are not aligned with one another, while they are aligned in other embodiments.

FIG. 6 illustrates a perspective, schematic view of an implantable sling assembly 70 or sling/sheath/sleeve combination of the disclosed invention that may be placed and positioned during an interventional procedure using the instruments described herein. Sling assembly 70 includes a sling 75 comprising a length of biocompatible material such as surgical mesh extending substantially between first sleeve 72 and second sleeve 74. The sling 75 may comprise any type of natural or synthetic biocompatible material, including woven and non-woven materials and various types of porous materials. Many types of surgical mesh compositions are known in the art and are suitable for use in the sling assembly 70 of the disclosed invention.

The term “sling,” as used herein, encompasses any structure that may be placed or implanted using instruments of the disclosed invention. In one embodiment, the sling 75 comprises a length of porous material, such as a synthetic surgical mesh material having a generally longitudinal flat surface. Sling 75 may comprise, for example, a length of a polypropylene knitted monofilament mesh material having the warp, or the direction of minimum stretch, aligned in the direction of its length. In one embodiment, the longitudinal edges of sling 75 are sealed or otherwise treated to prevent the protrusion of loose fibers and to provide generally smooth edges. In alternative embodiments, sling 75 may comprise a variety of biocompatible materials that are suitable for placement supporting an internal physiological structure. In general, the sling 75 comprises a porous material that permits, or facilitates, cellular and tissue in-growth to maintain and fix placement of the sling. The sling 75 may be associated with various agents that facilitate bonding, healing, tissue growth, or the like, as is well known in the art.

The length and width of the sling 75 may vary, depending on the interventional application and the placement environment. The width of sling 75 is generally from about 5 mm to about 15 mm, with a width of from about 8 mm to about 12 mm being preferred for many embodiments. The length of the sling 75 is generally from about 30 to about 60 cm, with a length of from about 35 to about 50 cm being preferred for many embodiments. The thickness of the sling 75 is generally from about 0.20 to about 1.0 mm, with a thickness of from about 0.60 to about 0.90 being preferred for many embodiments.

Each end of the sling 75 may be attached or attachable to, or mountable or mounted on, or associated with, an insertion member, such as a first and/or second sleeve 72, 74. The attachment or association between the sling and the sleeve(s) may be detachable, or it may be permanent or semi-permanent using various attachment mechanisms such as bonding, thermo-forming, welding, stitching, and the like. For some applications, the sling 75 is used and placed simply using the associated insertion members or sleeves, which are illustrated as generally tubular members in FIG. 6 and described in greater detail below.

In another embodiment, the sling 75 is substantially enclosed by an overlying sheath 76, which comprises a length of biocompatible material extending substantially between first sleeve 72 and second sleeve 74. In some embodiments, the sheath 76 comprises a biocompatible polyethylene material provided as a sheet material formed as a substantially flat “envelope” or covering that substantially surrounds and encloses the sling 75 substantially along its length. Many different biocompatible materials are known in the art for use as sheath materials. In one embodiment, the sheath comprises a linear low density polyethylene tubing material having a width of about 3.5-6 cm and a thickness of about 70μ.

The sheath 76 is sized and configured to slide freely with respect to the underlying sling 75 and may be formed as a substantially continuous envelope along the length of the sling 75. Alternatively, the sheath 76 may be formed as two pieces that meet or overlap and are slidable with respect to one another. In one embodiment, for example, sheath 76 is formed as two envelope-like sections that meet and, optionally, overlap one another in a telescoping arrangement at a generally central location 78 along the length of the sheath and sling. In alternative embodiments, a sheath formed of two sections may meet and, optionally, overlap one another at a location along the length of the sheath and sling that is offset from a central location.

The sling and/or sheath may have a tapered configuration in the terminal end region(s), as illustrated in FIG. 6, as they approach the attachment to sleeves 72 and 74. Terminal ends of either, or both, the sling and sheath are attached at or near the ends of sleeves 72 and 74.

Sleeves 72, 74 may be provided as generally cylindrical structures having a generally constant diameter along their lengths. Alternatively, the sleeves may be tapered toward either a terminal distal end, or toward a sling association end, or the sleeves may taper in both directions. During an interventional procedure, one or both of the sleeves are simultaneously or sequentially associated with an instrument to place, or implant, the sling. The sleeves have an internal cavity or passage 73 provided along at least a portion of their length for insertion of an instrument, such as the needle of an insertion instrument of the present invention. Instruments may be inserted into passage(s) 73 from openings at the distal end of each sleeve. Alternatively, slots or openings may be provided in the sleeves to provide access to the sleeve internal passage(s) for association with instruments.

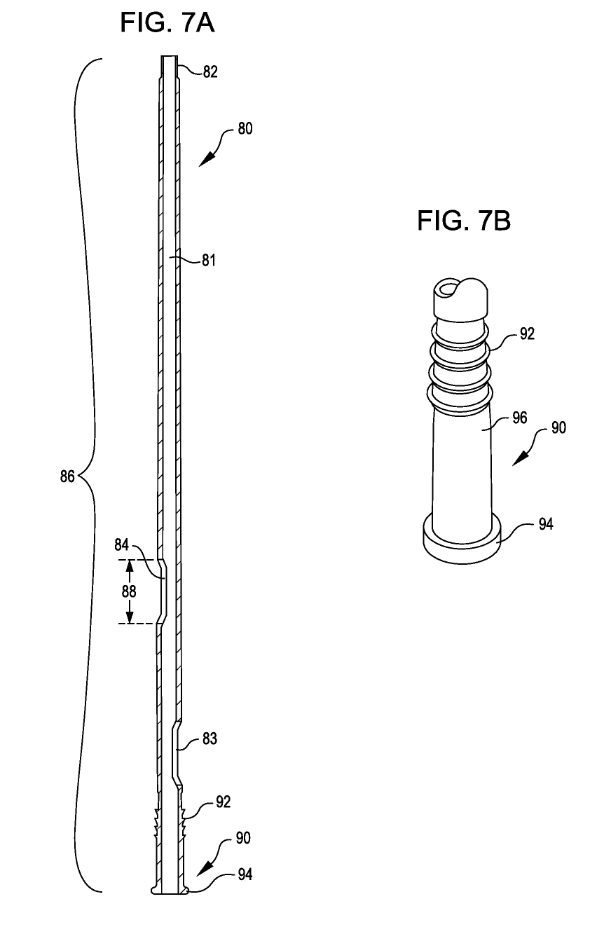

FIG. 7A shows another embodiment of a sleeve 80 of the present invention. Sleeve 80 is preferably formed as a generally tubular structure from a material that is resilient and at least somewhat flexible. UV-resistant polypropylene, heat-shrinking tubing materials, and other flexible plastic and polymeric materials are suitable for construction of sleeve 80. Huntsman PE 2053 (LDPE) USP Class VI material is an exemplary material for construction of the sleeve(s) of the disclosed invention. The sleeves may be reinforced if desired, as is known in the art.

In the embodiment shown in FIG. 7A, sleeve 80 is generally cylindrical and provides a central passage 81 for insertion of an instrument, such as the needle portion of an insertion instrument. The overall length 86 of sleeve 80 measures from about 15 cm to about 22 cm and, according to one embodiment, is from about 18.5 to about 19.7 cm. Sleeve 80 has a distal tapered region 82 having a smaller inner diameter cavity or passage than that of other portions of the sleeve 80. The distal tapered region 82 provides a tighter frictional fit when instruments, such as needle instruments, are mounted through the sleeve 80 and through the distal tapered region 82. Both the outer and inner diameters of the sleeve 80 may be substantially constant along the length of the sleeve, or may be tapered. The sleeve wall may have a substantially constant thickness, or the thickness of the sleeve wall may vary over the length of the sleeve. The outer diameter of the sleeve 80 may be varied, for example, to vary the thickness of the sleeve wall and increase, or decrease, the thickness in proximity to the region that is attached to the sling and/or sheath. The outer diameter of the sleeve 80 may be reduced, for example, in a region extending from the terminal end to provide enhanced flexibility for ease of needle insertion and removal. The inside diameter of the sleeve(s) may be similar in dimension and cross-sectional configuration to the outer diameter of a needle instrument to be used for placement of the sling, so that the needles or portions of the needles may be inserted into and maintained in the sleeves by frictional contact with the inner surfaces of the sleeves.

The sleeve is preferably provided with at least one slot opening to its inner passage for insertion of a needle instrument through the slot and into the inner passage of the sleeve, or for positioning a needle instrument passing through the inner passage of the sleeve through the slot to the exterior of the sleeve. In some embodiments, at least one slot is provided in each sleeve and, in some embodiments, multiple slots are provided in at least one of the sleeves. Slots may be aligned and oriented so that they open on a surface aligned with one of the “flat” surfaces of a sling/sheath combination. Alternatively, one or more slots may be aligned and oriented so that they open in a direction that is aligned with one of the “edges” of the sheath. In yet additional embodiments, one or more slots may be aligned and oriented at an angle to a flat surface or edge of the sheath. Multiple slots provided on a sleeve may be oriented to open on radially opposing surfaces of the sleeve.

Slots for insertion of needle instruments are generally provided in a proximal area of the sleeve although, in some embodiments, insertion slots may alternatively or additionally be located in a distal portion of the sleeve(s). In some embodiments, a sleeve member has at least one slot oriented facing one of the flat surfaces of the sling/sheath combination and at least one additional slot oriented facing another of the flat surfaces of the sling/sheath combination. This arrangement permits insertion of an instrument on either side of the sleeve member. The slot opening(s) preferably have a generally elongated configuration and may have a generally rectangular configuration having rounded ends.

In the embodiment shown in FIG. 7A, sleeve 80 is provided with two elongated slots 83 and 84 facing generally opposite directions (e.g., located on substantially opposed radial surfaces). Each of the elongated slots is oriented, as described above, generally with one of the major (flat) surfaces of the sling or the sling/sheath assembly. Slot 83 is located in proximity to an interface region 90 of the sleeve 80 that is attached to or associated with the sling or the sling/sheath assembly during use. Slot 84 is located further from the interface region 90 toward the distal region of the sleeve 80. Both of the slots 83, 84 are preferably provided closer to interface region 90 than distal end 82. The distance between slots 83 and 84 is preferably at least about the length of one of the slots and is preferably less than the length of three slots. In one exemplary embodiment, the total length of the sleeve is about 19 cm; there are two slots, each having a length 88 of about 12 mm; and the distance between the distal end of slot 83 and the proximal end of slot 84 is about is about 2.2 cm. The term “about,” as used herein, contemplates variances of up to +/−20% of the relevant dimension or other parameter.

FIG. 7B illustrates a preferred embodiment of an interface region 90 of sleeve 80. Interface region 90 is provided at a proximal end of the sleeve where the association with the sling and/or the sling/sheath assembly takes place. In the embodiment illustrated, sleeve interface region 90 is provided with a plurality of ridges or barbs 92 and a collar 94 at the proximal terminal end of the sleeve. Collar 94 has a larger outer diameter than the outer diameter of the cylindrical body 96 of interface region 90. The outer and/or inner surfaces of interface region 90 may have a generally cylindrical configuration with a generally constant diameter, or may have a generally tapered configuration, such that the proximal area of interface region 90 has a larger diameter than the distal area of interface region 90. A distal area of interface region 90 is provided with one or more upstanding ridges 92. Ridges 92 are generally circular and oriented generally transverse to the longitudinal axis of sleeve 80 in the embodiment illustrated in FIG. 7B; in alternative embodiments, ridges 92 may have a curved profile or be oriented in an angular orientation other than transverse to the longitudinal axis of sleeve 80. Ridges 92 may have a generally flat surface oriented toward the distal end of sleeve 80 and an angled or curved surface oriented toward the proximal end of sleeve 80 and collar 94, as shown in FIGS. 7A and 7B.

The terminal portions of a sling or a sling/sheath assembly are attached to or associated with the proximal portions of one or more sleeves prior to use in an interventional procedure. It is important that the sling and sleeve are attached to or associated with one another during use to provide a generally high breaking strength and to prevent the sling from being detached from the sleeve inadvertently during an interventional procedure. In preferred embodiments described herein, the terminal portions of a sling or sling/sheath assembly are bonded to the proximal portions of sleeves prior to packaging so that the medical professional can use the sling/sheath/sleeve assembly directly with insertion instruments.

The surfaces of terminal portions of the sling or the sling/sheath assembly may be attached by bonding or other fastening mechanisms to proximal surfaces of the respective sleeves. Terminal portions of the sling or the sling/sheath assembly may, for example, be heat welded to the sleeve(s) or mounted on the sleeves using an intermediate structure, such as a band or a short segment of tube or heat-shrinkable material. In one embodiment, the sling or sling/sheath assembly is mounted to sleeves at each terminal end by way of interface region 90, such that a terminal portion of the sling/sheath assembly is contacted to the outer surface of interface region 90, covering the ridges 92 and collar 94. Various attachment mechanisms such as bonding, thermo-forming, welding, stitching, and the like, may then be used to permanently attach the sling or the sheath, or both, to the interface region 90 of sleeve 80. In one embodiment, the terminal portion of the sling or sheath, or both, is thermally bonded to the sleeve interface region 90 using a heat shrinkable tubing segment. The breaking strength of the sling and sleeve assembly in the longitudinal direction when the sling and sleeve are bonded or associated is preferably at least 48 newtons.

The sleeves and needle instruments are preferably sized and configured to provide frictional sliding of the inner passages of the sleeves over the outer surfaces of the needle instruments for placement of the sling assembly on needles and removal of the combination from the needles. The dimensional tolerances are sufficiently close, and/or the material forming the sleeves is sufficiently “sticky” with respect to the instruments to maintain the sleeves in a desired a mounting position during an operation. One or both of the sleeves may have one or more openings or slots facilitating mounting of an insertion instrument, as described above.

One or both sleeves may be mounted on an insertion instrument by passing a needle component of the instrument through a terminal end of the sleeve and into the sleeve passageway. In this system, slots may be provided for the needles that inserted through the terminal end of the sleeve to exit the sleeve after an appropriate length of the sleeve has been mounted on the insertion instrument. Alternatively, one or both sleeves may be mounted on an insertion instrument by passing a needle component of the instrument through a slot and into the sleeve passageway, allowing the needle component to exit the sleeve through a terminal end of the sleeve after an appropriate length of the sleeve has been mounted on the insertion instrument. In an alternative embodiment, one or both sleeves may be mounted on an insertion instrument by passing a needle component of the instrument through a first slot and into the sleeve passageway, and allowing the needle component to exit the sleeve through a second slot in the sleeve after an appropriate length of the sleeve has been mounted on the insertion instrument.

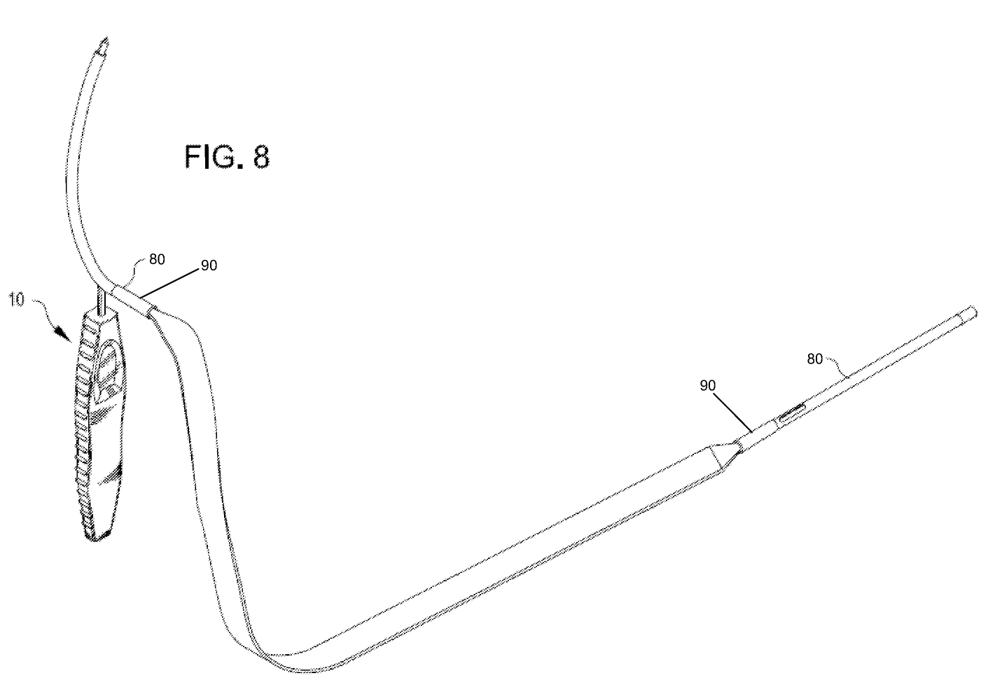

FIG. 8 illustrates a long, gently curved needle instrument 10 of the disclosed invention inserted through a slot in sleeve 80. The terminal end of the needle is inserted into the slot and the inner surface of the sleeve is slid over the needle portion and retained in place on the needle instrument. The fit between the inner surface of the sleeve and the outer surface of the needle instrument is preferably a light friction fit generated by sliding the tubular sleeve onto the curved needle. In one embodiment, an instrument of the disclosed invention, such as curved needle instrument 10, is inserted through a slot in the sleeve component, and the needle is moved in relation to the sleeve until the slot is positioned at or in proximity to the transition portion of the needle. The terminal end of the needle preferably protrudes from the terminal end of the sleeve when the sleeve is installed on the needle. In some embodiments, as described above, sleeve 80 is provided with multiple slots and, when multiple slots are provided, the curved needle instrument is generally inserted through the slot that provides a suitable length of sleeve mounted on the needle with the terminal needle end protruding from the sleeve. In the embodiment of sleeve 80 illustrated in FIG. 7A, the long, gently curved needle 10 is preferably inserted through the slot 83 located in proximity to the interface region 90 of the sleeve 80. The length of sleeve 80 between the insertion slot for needle instrument 10 and the distal end of the sleeve is substantially similar to and generally slightly less than the overall length of the curved section of needle instrument 10.

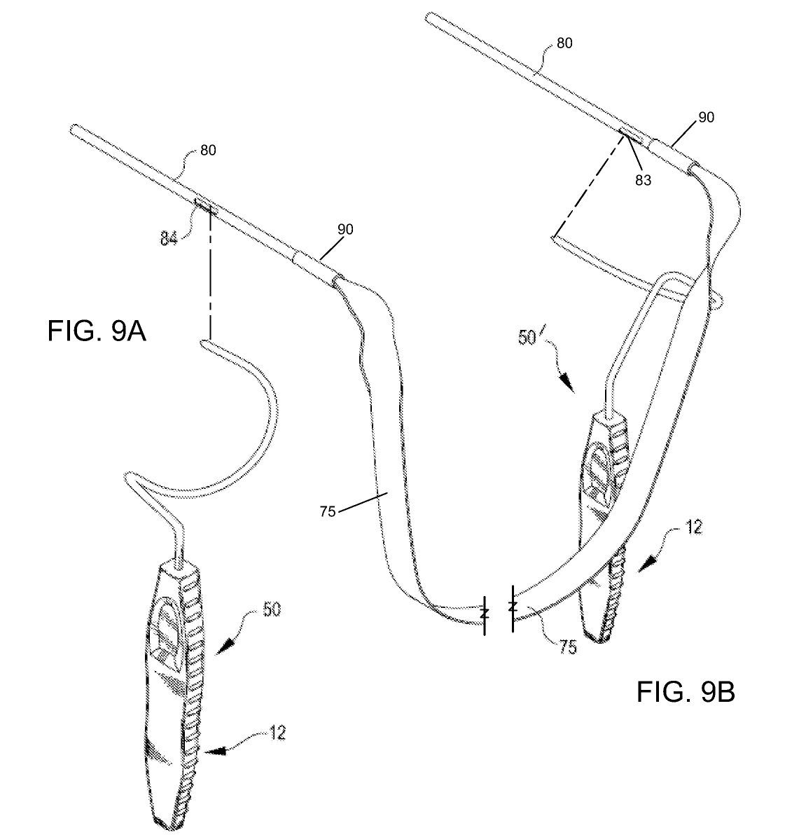

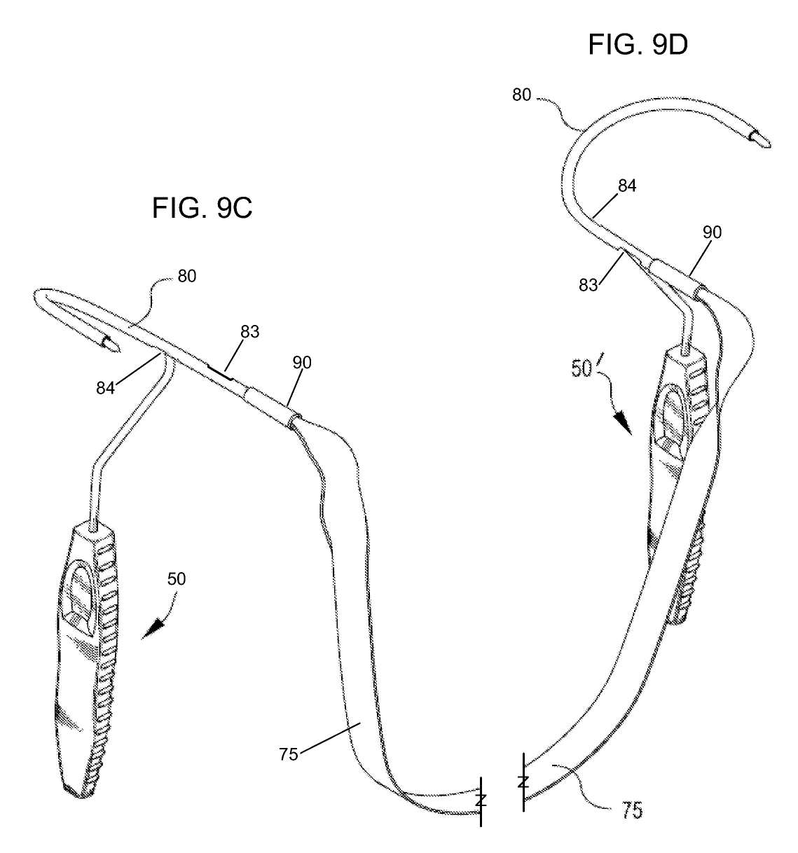

FIGS. 9A and 9B illustrate the placement of directional needle instruments 50 and 50′, respectively, of the disclosed invention for mounting on sleeves 80. The terminal ends of the needles of instruments 50 and 50′ are inserted through slots 84 and 83 in sleeves 80 respectively, and the sleeves 80 are slid along and mounted on the needles until the sleeves 80 are mounted on the needles as shown in one embodiment, in FIGS. 9C and 9D. In this embodiment, the terminal ends of a the directional needles are inserted through slots 84 and 83 in the sleeve component, respectively, and the needles are mounted through the sleeves 80 until the slots are positioned at or in proximity to the proximal ends of the curved portion of directional needle instruments 50 and 50′, as shown in FIGS. 9C and 9D. The terminal end of the needle preferably protrudes from the terminal end of the sleeve when the sleeve is installed on the needle. In some embodiments, as described above, in which sleeve 80 is provided with multiple slots, directional needles of instruments 50, 50′ are generally inserted through the slot that provides a suitable length of sleeve mounted on the curved portion of the needle, with the terminal needle end protruding from the sleeve. In the embodiment of sleeve 80 illustrated in FIG. 7A, directional needle of instrument 50 is preferably inserted through the slot 84 located more centrally with respect to the interface region 90 of the sleeve 80 than slot 83, and directional needle instrument 50′ is preferably inserted through the slot 83 located in proximity to interface region 90. The length of sleeve 80 between the insertion slot for directional needle instruments 50 and 50′ and the distal end of the sleeve is substantially similar to and generally slightly less than the overall length of the curved section and distal extension of directional needle instruments 50 and 50′.

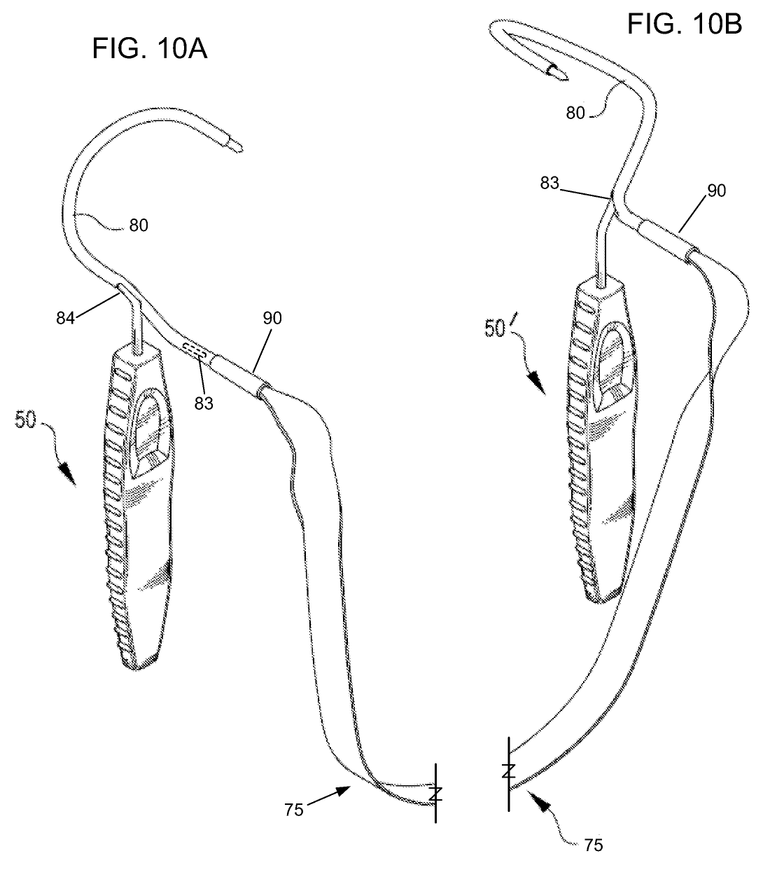

An alternative insertion and mounting configuration and procedure using directional needle instruments 50 and 50′ is illustrated in FIGS. 10A and 10B. In this embodiment, the distal end of the needle of instrument 50 is inserted through slot 84, and the distal end of the needle of instrument 50′ is inserted through slot 83. The distal ends of one or both of the needles of instruments 50 and 50′ are mounted on sleeves 80 through slots 84 and 83, respectively, such that they protrude from slots the distal ends of sleeves 80. The needles may be inserted into the sleeves 80 and thereby be mounted to the sling/sheath/sleeve combination prior to or during an operation.

Curved needle instruments 440 of the disclosed invention may also be inserted through a slot in sleeve 80. The terminal end of the needle is inserted into a slot in the sleeve 80, and the inner surface of the sleeve 80 is slid over the curved needle portion and retained in place on the needle instrument 440. In this embodiment, as the terminal end of the curved needle instrument 440 is inserted through a slot in the sleeve component 80, and the needle is moved in relation to the sleeve 80 until the slot is positioned at or in proximity to the transition portion of the needle. The terminal end of the needle preferably protrudes from the terminal end of the sleeve 80 when the sleeve 80 is installed on the needle. In some embodiments, as described above, in which sleeve 80 is provided with multiple slots, the curved needle instrument 440 is generally inserted through the slot that provides a suitable length of sleeve mounted on the needle with the terminal needle end protruding from the sleeve. In the embodiment of sleeve 80 illustrated in FIG. 7A, the curved needle instrument 440 is preferably inserted through the slot 84, which is located more centrally with respect to the interface region 90 of the sleeve 80 than slot 83. The length of sleeve 80 between the insertion slot for curved needle instrument 440 and the distal end of the sleeve is substantially similar to and generally slightly less than the overall length of the curved section of curved needle instruments 440.

Various sling assemblies may be mounted on various needle instruments in various configurations for use in various types of procedures. The needle instruments and sling assemblies of the disclosed invention are highly versatile and may be used in many different configurations for many different types of procedures. Various transition zones between different portions of the needle instruments, such as between a curved portion and a differently curved or straight portion of the needle, may serve as a marker zone that indicates a desirable positioning of the sleeve relative to the needle. Multiple needle components may be configured, for example, to have a transition zone, or “knee,” between a curved and straight section, that indicates proper positioning of a terminal end of the sleeve and determines how far the needle is pushed into the sleeve. Transition zones or mounting positions may alternatively be provided by visible indicators such as colored bands, marks, or the like. In general, the needle insertion instruments with the sleeves mounted thereon may be used in an interventional procedure without requiring stylets or catheter guides.

Kits comprising combinations of the insertion instruments and sling/sheath/sleeve assemblies may be assembled. The kits preferably comprise at least two insertion instruments in combination with a sling or sling/sheath or sling/sheath/sleeve assembly of the present invention. In one embodiment, a kit of the present invention comprises a long, gently curved insertion instrument and a shorter, bi-directional curved insertion instrument in combination with a sling or sling/sheath or sling/sheath/sleeve assembly of the present invention. In another embodiment, a kit of the present invention comprises a long, gently curved insertion instrument and a pair of complementary, directional curved needle instruments in combination with a sling or sling/sheath or sling/sheath/sleeve assembly of the present invention. In another embodiment, a kit of the present invention comprises a shorter, bi-directional curved insertion instrument and a pair of complementary, directional curved needle instruments in combination with a sling or sling/sheath or sling/sheath/sleeve assembly of the present invention. In yet another embodiment, a kit of the present invention comprises a pair of complementary, directional curved needle instruments in combination with a sling or sling/sheath or sling/sheath/sleeve assembly of the present invention.

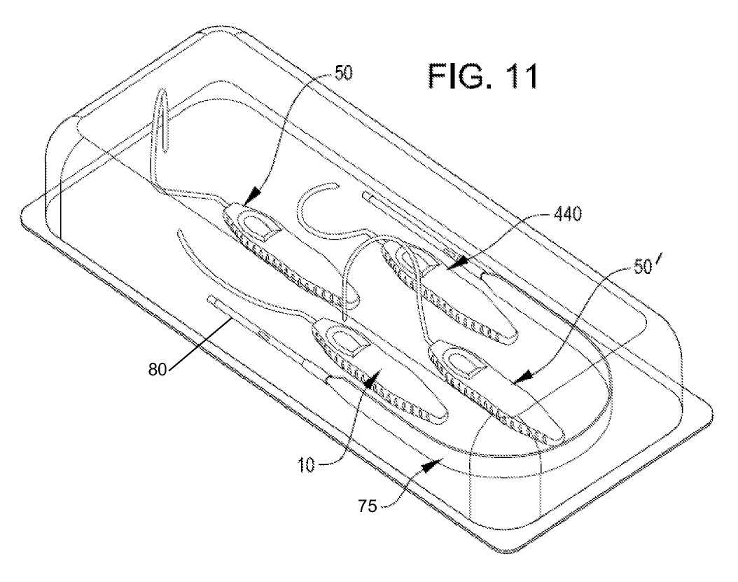

FIG. 11 illustrates a kit of the disclosed invention comprising a needle instrument 10, a needle instrument 440, two complementary, directional needle instruments 50, 50′, a sling 75 for supporting an internal physiological structure and two sleeves 80 packaged in a sealable packaging unit. This kit is a particularly useful combination, since it provides considerable flexibility to a surgeon in the type of procedure that may be performed using various components of the kit. Various combinations and numbers of needle instruments may be provided with various sizes, shapes and configurations of sling/sheath/sleeve combinations in kits of the disclosed invention. The instruments and implantable devices and combinations are preferably provided in a sterile form and are packaged in a sealed, sterile package for use in surgical and minimally invasive surgical procedures. In many embodiments, the instruments forming the kit combinations are intended for single use. Instructional materials may also be provided with the kit(s) and/or their components.

Methods for performing interventional procedures and placing sling assemblies of the present invention as a suburethral and/or bladder neck slings are straightforward. One or more suitable insertion instruments is selected from a kit, and the needle portion of the instrument is advanced through the sleeve mounted on or associated with the sling assembly until the distal end of the needle protrudes from the distal end of the sleeve. Both ends of the sling assembly are then inserted in the patient at desired incisions or locations, using the insertion instruments, depending on the surgical placement method chosen. The insertion instruments are then manipulated so that the sling assembly forms a loop beneath the urethra, and the insertion instruments are removed following successful placement of the sling assembly. The sling assembly is then removed from one or both of the insertion instruments by pushing from the proximal end of the sleeve/sheath/mesh assembly toward the distal end of the insertion needle. Once the sleeve/sheath/mesh assembly is advanced beyond the distal end of the needle, it can be manually pulled off the needle by grasping the distal end of the sleeve.

The sling may then be adjusted by pulling outwardly on one or both of the sleeves, so that the sling material is placed appropriately below the urethra. The sling assembly may then be tensioned, if desired and to the degree desired by the surgeon or other medical professional. The sheath/sling ends are then separated (e.g. by cutting) from the sleeves in the sleeve interface region to remove each associated sleeve from the sling assembly, and to expose each sling and sheath end. The sheath may be removed by grasping each of the exposed sheath ends independent of the sling and pulling outwards with equal tension on each sheath. Stabilizing the sheath and sling material under the urethra facilitates the removal of the sheath. The tension and placement of the sling may be verified and adjusted, as desired, and then the distal ends of the sling are removed (e.g. by cutting) so that the sling ends retract below the skin incisions. Incisions may be closed according to standardized methods.

The disclosed invention has been described with reference to specific embodiments and figures. These specific embodiments should not be construed as limitations on the scope of the invention, but merely as illustrations of exemplary embodiments. It is further understood that many modifications, additions and substitutions may be made to the described instruments, components and kits without departing from the scope of the disclosed invention.

I claim:

1. An insertion instrument for implanting a biocompatible sling comprising: an insertion needle having a curved needle portion mounted at a distal end of a handle, wherein the handle has opposing side surfaces that are convexly raised with respect to adjacent grip surfaces, and each of the opposing side surfaces is provided with a recess having a generally thumb-like configuration positioned toward the center of the handle from the distal end of the handle.

2. An insertion instrument of claim 1, wherein the overall length of each recess is at least about 10% of the total length of each side surface.

3. An insertion instrument of claim 1, wherein the opposing side surfaces have generally narrower, tapered portions in proximity to distal and proximal ends and generally wider portions at distal and proximal end regions.

4. An insertion instrument of claim 1, wherein the dimensions of the distal end and a proximal end of the handle are substantially the same.

5. An insertion instrument of claim 1, wherein the respective side surfaces and the respective grip surfaces are aligned symmetrically on a central longitudinal axis of the handle.

6. An insertion instrument of claim 1, wherein each of the grip surfaces has a plurality of raised elements oriented substantially transverse to a central longitudinal axis of the handle.

7. An insertion instrument of claim 1, wherein the curved needle portion of the insertion needle forms an arc of from about 60° to about 70° and a proximal end and a distal end of the curved needle portion terminate substantially on a central longitudinal axis of the handle.

8. An insertion instrument of claim 1, wherein the curved needle portion of the insertion needle curves along a generally constant radius around a center point located on a central longitudinal axis of the handle.

9. An insertion instrument of claim 1, wherein a distal terminal end of the curved needle portion extends past a centerline formed by the central longitudinal axis and forms an arc of from about 20° to about 70° beyond the centerline.

10. An insertion instrument of claim 1, wherein the curved needle portion is substantially planar and is aligned on a plane that substantially bisects the handle side surfaces.

11. An insertion instrument of claim 1, wherein the insertion needle has a transition section aligned substantially on a central longitudinal axis of the handle and a curved needle section having an angled portion that transitions into a curved portion and terminates at a distal terminal end.

12. An insertion instrument of claim 1, wherein the axis of the angled portion extends at an angle of from about 10° to about 80° from a central axis of the transition section.

13. An insertion instrument of claim 1, wherein the curved portion of the curved needle section curves through an arc of about 180° and has a distal extension of the curved needle section that extends beyond the axis of the angled portion and terminates at the distal terminal end.

14. An insertion instrument of claim 1, wherein the handle comprises a reusable, sterilizable material.

15. An insertion instrument of claim 1, wherein the handle comprises a reusable, autoclavable material.
